(19) United States
(12) Patent Application Publication
(10) Pub. No.: US 2021/0244360 A1
(43) Pub. Date: Aug. 12, 2021

(54) SENSING, MAPPING, AND THERAPY CATHETER WITH MULTIPLE CATHETERLETS

(71) Applicant: St. Jude Medical, Cardiology Division, Inc., St. Paul, MN (US)

(72) Inventor: Eric S. Olson, Maplewood, MN (US)

(21) Appl. No.: 16/972,581

(22) PCT Filed: Jun. 6, 2019
(86) PCT No.: PCT/IB2019/054739

§ 371 Date: Dec. 5, 2020

Related U.S. Application Data
(60) Provisional application No. 62/681,928, filed on Jun. 7, 2018.

(51) Int. Cl. A61B 5/00; A61B 5/287; A61B 18/14

(52) U.S. Cl. CPC A61B 5/6859; A61B 5/287; A61B 5/367; A61B 5/6885; A61B 2018/1467; A61B 18/1492

(57) ABSTRACT

One or more embodiments of the present disclosure are directed to a catheter including a plurality of flexible catheterlets, in such an embodiment each of the catheterlets may include an electrode proximate a distal end of the catheterlet. In some more specific embodiments, one or more of the catheterlets may be free of electrodes at a distal most end.

### CROSS-REFERENCE TO RELATED APPLICATIONS

This application claims the benefit of U.S. provisional application No. 62/681,928, filed 7 Jun. 2018, which is hereby incorporated by reference as though fully set forth herein.

### BACKGROUND

### a. Field

This disclosure relates to systems, methods, and apparatuses for intravascular catheter-based cardiac electrophysiology mapping and ablation therapy.

### b. Background Art

Electrophysiology catheters are used in a variety of diagnostic and/or therapeutic medical procedures to diagnose and correct atrial arrhythmias, including for example, ectopic atrial tachycardia, atrial fibrillation, and atrial flutter. Arrhythmias may create a variety of dangerous conditions including irregular heart rates, loss of synchronous atrioventricular contractions and stasis of blood flow which can lead to a variety of ailments and even death.

Typically in intravascular catheter procedures, a catheter is manipulated through a patient's vasculature to, for example, a patient's heart where a distal tip of the catheter may be used for mapping, ablation, diagnosis, etc. Once at the intended site, treatment may include radio frequency (RF) ablation, cryoablation, lasers, chemicals, high-intensity focused ultrasound, etc., to create a lesion in the cardiac tissue. This lesion disrupts undesirable electrical pathways and thereby limits or prevents stray electrical signals that lead to arrhythmias. As readily apparent, such treatment requires precise control of the catheter during manipulation to and at the treatment site.

To position a catheter at a desired site within the body, mechanical steering features may be incorporated into the catheter (or an introducer), allowing medical personnel to manually manipulate the catheter.

In order to facilitate the advancement of catheters through a patient's vasculature, a navigating system may be used (e.g., electric-field-based and navigation systems) to determine the position and orientation of the catheter within the body.

Various therapies may be delivered by intravascular catheters to tissue with varied surface geometry. To better accommodate variations in tissue surface geometry and to provide contiguous contact with the tissue for therapy, it may be desirable to have multiple flexible elements at a distal end of the catheter, each of the flexible elements being capable of mapping and ablating the respective contacted tissue.

The foregoing discussion is intended only to illustrate the present field and should not be taken as disavowal of claim scope.

### BRIEF SUMMARY

Aspects of the present disclosure are directed to a catheter including a plurality of catheterlets. Each of the catheterlets having a proximal end and a distal end, and an electrode. Each electrode is proximate the respective catheterlet distal end, and the plurality of catheterlets are flexible.

In one embodiment of the present disclosure, a catheter is disclosed including a plurality of catheterlets, each with a proximal end and a distal end. Each catheterlet includes a first electrode and a second electrode, where the first electrode is proximate the distal end and the second electrode is proximal of the first electrode. In a deployed position, the first electrode and the second electrode or a respective catheterlet are separated by an angled portion.

In another embodiment, a catheter is disclosed including a plurality of compound catheterlets, each of the plurality of compound catheterlets having a first portion with a first longitudinal axis, a second portion with a second longitudinal axis, and a third portion with a third longitudinal axis. The catheter has a deployed position and an undeployed position; wherein, in the undeployed position, the first longitudinal axis, the second longitudinal axis, and the third longitudinal axis are substantially aligned with the catheter longitudinal axis. In the deployed position, the first longitudinal axis is substantially aligned with the catheter longitudinal axis, and the first longitudinal axis is angled relative to the second longitudinal axis, and the second longitudinal axis is angled relative to the third longitudinal axis when the plurality of compound catheterlets are partially or fully extended from a sheath. In some embodiments, each of the plurality of compound catheterlets may be unsecured at a distal end.

In another embodiment, a catheter includes a central catheterlet with an electrode, and a plurality of peripheral catheterlets. Each peripheral catheterlet having an electrode, and is positioned around the central catheterlet.

### BRIEF DESCRIPTION OF THE DRAWINGS

FIG. 1 is a system diagram showing a medical device and a medical positioning system, in accordance with various embodiments of the present disclosure.

FIG. 2A is a cross-sectional plan view of a catheter with multiple catheterlets positioned within a sheath of the catheter, in accordance with various embodiments of the present disclosure.

FIG. 2B is a cross-sectional plan view of a catheter with multiple catheterlets positioned within a sheath of the catheter and surrounding a central catheter, in accordance with various embodiments of the present disclosure.

FIG. 3A is a side view of a distal end of the catheter of FIG. 2A with multiple layers of catheterlets deployed, in accordance with embodiments of the present disclosure.

FIG. 3B is a side view of a distal end of the catheter of FIG. 2A with multiple layers of catheterlets deployed, in accordance with various embodiments of the present disclosure.

FIG. 3C is an isometric side view of a distal end of a catheter with multiple catheterlets deployed, in accordance with various embodiments of the present disclosure.

FIG. 3D is an isometric side view of a distal end of a catheter with multiple catheterlets deployed, in accordance with various embodiments of the present disclosure.

FIG. 3E is a side view of a distal end of a catheter with multiple interleaved catheterlets deployed, in accordance with various embodiments of the present disclosure.

FIG. 4 is an isometric side view of a distal end of a catheter with multiple catheterlets surrounding a central catheterlet, in accordance with various embodiments of the present disclosure

FIG. 5 is an isometric side view of a distal end of a catheter with a plurality of compound catheterlets deployed thereon, in accordance with various embodiments of the present disclosure.

### DETAILED DESCRIPTION OF EMBODIMENTS

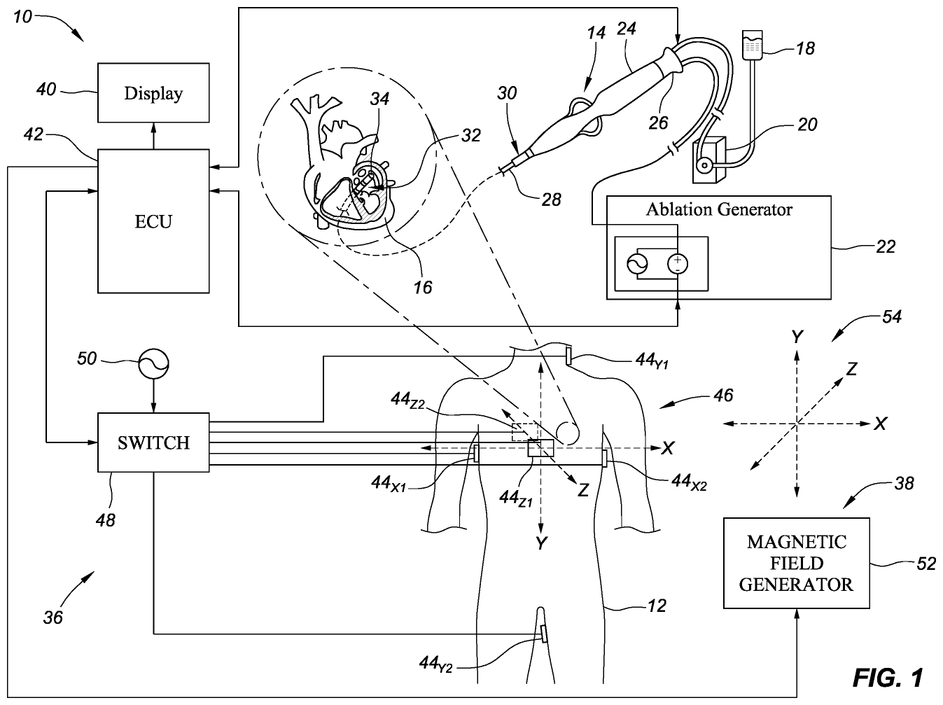

Referring now to the figures, in which like reference numerals refer to the same or similar features in the various views, FIG. 1 illustrates one embodiment of a system 10 for navigating a medical device within a body 12. In the illustrated embodiment, the medical device comprises a catheter 14 that is shown schematically entering a heart that has been exploded away from the body 12. The catheter 14, in this embodiment, is depicted as an irrigated radiofrequency (RF) ablation catheter for use in the treatment of cardiac tissue 16 in the body 12. It should be understood, however, that the system 10 may find application in connection with a wide variety of medical devices used within the body 12 for diagnosis or treatment. For example, the system 10 may be used to navigate an electrophysiological mapping catheter, an intracardiac echocardiography (ICE) catheter, or an ablation catheter using a different type of ablation energy (e.g., cryoablation, ultrasound, etc.). Further, it should be understood that the system 10 may be used to navigate medical devices used in the diagnosis or treatment of portions of the body 12 other than cardiac tissue 16. Further description of the components of system 10 are contained in U.S. patent application Ser. No. 13/839,963 filed on 15 Mar. 2013, which is hereby incorporated by reference in its entirety as though fully set forth herein.

Referring still to FIG. 1, the ablation catheter 14 is connected to a fluid source 18 for delivering a biocompatible irrigation fluid such as saline through a pump 20, which may comprise, for example, a fixed rate roller pump or variable volume syringe pump with a gravity feed supply from fluid source 18 as shown. The catheter 14 is also electrically connected to an ablation generator 22 for delivery of RF energy. The catheter 14 may include a handle 24; a cable connector or interface 26 at a proximal end of the handle 24; and a shaft 28 having a proximal end 30, a distal end 32, and one or more electrodes 34. The connector 26 provides mechanical, fluid, and electrical connections for conduits or cables extending from the pump 20 and the ablation generator 22. The catheter 14 may also include other conventional components not illustrated herein such as a temperature sensor, additional electrodes, and corresponding conductors or leads.

The handle 24 provides a location for the physician to hold the catheter 14 and may further provide means for steering or guiding the shaft 28 within the body 12. For example, the handle 24 may include means to change the length of one or more pull wires extending through the catheter 14 from the handle 24 to the distal end 32 of shaft 28. The construction of the handle 24 may vary.

The shaft 28 may be made from conventional materials such as polyurethane and may define one or more lumens configured to house and/or transport electrical conductors, fluids, or surgical tools. The shaft 28 may be introduced into a blood vessel or other structure within the body 12 through a conventional introducer. The shaft 28 may then be steered or guided through the body 12 to a desired location such as the tissue 16 using guide wires or pull wires or other means known in the art including remote control guidance systems. The shaft 28 may also permit transport, delivery, and/or removal of fluids (including irrigation fluids and bodily fluids), medicines, and/or surgical tools or instruments. It should be noted that any number of methods can be used to introduce the shaft 28 to areas within the body 12. This can include introducers, sheaths, guide sheaths, guide members, guide wires, or other similar devices. For ease of discussion, the term introducer will be used throughout.

The system 10 may include an impedance-based positioning sub-system 36, a magnetic-field-based positioning sub-system 38, a display 40, and an electronic control unit (ECU) 42 (e.g., a processor). Each of the exemplary system components is described further below.

The impedance-based positioning sub-system 36 and the magnetic-field-based positioning sub-system 38 are provided to determine the position and orientation of the catheter 14 and similar devices within the body 12. The position and orientation of the catheter 14 and similar devices within the body 12 can be determined by the sub-system 36 and/or the sub-system 38. The sub-system 36 may comprise, for example, the EnSite™ NavX™ system sold by St. Jude Medical, Inc. of St. Paul, Minn., and described in, for example, U.S. Pat. No. 7,263,397 titled “Method and Apparatus for Catheter Navigation and Location Mapping in the Heart,” the entire disclosure of which is hereby incorporated by reference as though fully set forth herein. The sub-systems 36 and 38 may comprise, for example, the EnSite Precision™ system sold by St. Jude Medical, Inc., of St. Paul, Minn. The sub-system 36 operates based upon the principle that when low amplitude electrical signals are passed through the thorax, the body 12 acts as a voltage divider (or potentiometer or rheostat) such that the electrical potential or field strength measured at one or more electrodes 34 on the catheter 14 may be used to determine the position of the electrodes, and, therefore, of the catheter 14, relative to a pair of external patch electrodes using Ohm's law and the relative location of a reference electrode (e.g., in the coronary sinus).

In the configuration shown in FIG. 1, the impedance-based positioning sub-system 36 includes three pairs of patch electrodes 44, which are provided to generate electrical signals used in determining the position of the catheter 14 within a three-dimensional coordinate system 46. The patch electrodes 44 may also be used to generate electrophysiology data regarding the tissue 16. To create axes-specific electric fields within body 12, the patch electrodes are placed on opposed surfaces of the body 12 (e.g., chest and back, left and right sides of the thorax, and neck and leg) and form generally orthogonal x, y, and z axes. A reference electrode/patch (not shown) is typically placed near the stomach and provides a reference value and acts as the origin of the coordinate system 46 for the positioning sub-system.

In accordance with the impedance based positioning sub-system 36 as depicted in FIG. 1, the patch electrodes include right side patch 44_{X1}, left side patch 44_{X2}, neck patch 44_{Y1}, leg patch 44_{Y2}, chest patch 44_{Z1}, and back patch 44_{Z2}; and each patch electrode is connected to a switch 48 (e.g., a multiplex switch) and a signal generator 50. The patch electrodes 44_{X1}, 44_{X2} are placed along a first (x) axis; the patch electrodes 44_{Y1}, 44_{Y2} are placed along a second (y) axis, and the patch electrodes 44_{Z1}, 44_{Z2} are placed along a third (z) axis. Sinusoidal currents are driven through each pair of patch electrodes, and voltage measurements for one or more position sensors (e.g., ring electrodes 34 or a tip electrode located near a distal end 32 of catheter shaft 28) associated with the catheter 14 are obtained. The measured voltages are a function of the distance of the position sensors from the patch electrodes. The measured voltages are compared to the potential at the reference electrode, and a position of the position sensors within the coordinate system 46 of the sub-system is determined.

The magnetic-field-based positioning sub-system 38 in embodiment of FIG. 1 employs magnetic fields to detect the position and orientation of the catheter 14 within the body 12. The sub-system 38 may include the MediGuide, Ltd. GMPS system, and generally shown and described in, for example, U.S. Pat. No. 7,386,339 titled “Medical Imaging and Navigation System,” the entire disclosure of which is hereby incorporated by reference as though fully set forth herein. In such a magnetic-field based sub-system, a magnetic field generator 52 with three orthogonally arranged coils (not shown) creates a magnetic field within the body 12 and controls the strength, orientation, and frequency of the field. The magnetic field generator 52 may be located above or below the patient (e.g., under a patient table), or in another appropriate location. Magnetic fields are generated by the coils, and current or voltage measurements for one or more position sensors (not shown) associated with the catheter 14 are obtained. The measured currents or voltages are proportional to the distance of the sensors from the coils, thereby facilitating determination of a position of the sensors within a coordinate system 54 of sub-system 38.

The display 40 is provided to convey information to a physician to assist in diagnosis and treatment. The display 40 may comprise one or more conventional computer monitors or other display devices. The display 40 may present a graphical user interface (GUI) to the physician. The GUI may include a variety of information including, for example, an image of the geometry of the tissue 16, electrophysiology data associated with the tissue 16, graphs illustrating voltage levels over time for various electrodes 34, and images of the catheter 14 and other medical devices and related information indicative of the position of the catheter 14 and other devices relative to the tissue 16.

The ECU 42 provides a means for controlling the operation of various components of the system 10, including the catheter 14, the ablation generator 22, and magnetic generator 52 of the magnetic-field-based positioning sub-system 38. The ECU 42 may also provide a means for determining the geometry of the tissue 16, electrophysiology characteristics of the tissue 16, and the position and orientation of the catheter 14 relative to tissue 16 and the body 12. The ECU 42 may also provide a means for generating display signals used to control the display 40.

As the catheter 14 moves within a body 12, and within the electric field generated by the electric-field-based positioning sub-system 36, the voltage readings from the electrodes 34 change indicating a location of catheter 14 within the electric field (and the coordinate system 46). The electrodes 34 may communicate position signals to ECU 42 through a conventional interface (not shown).

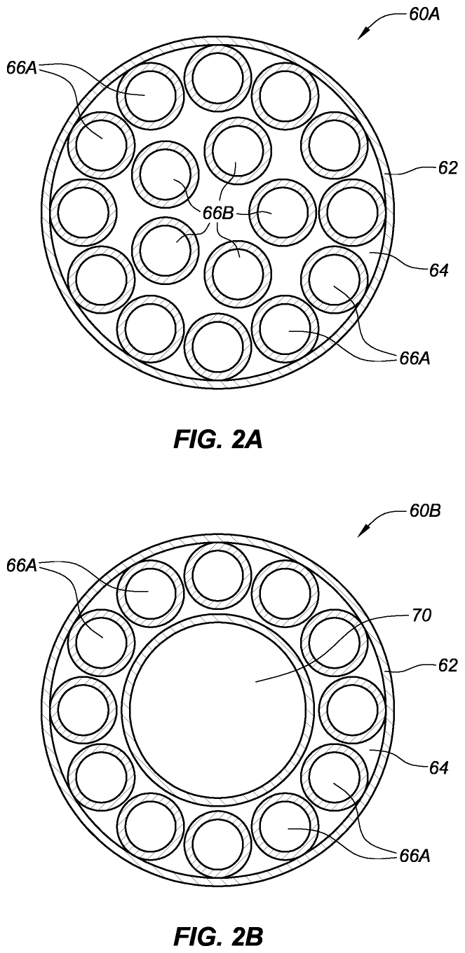

FIG. 2A is a cross-sectional plan view of multiple catheterlets of a catheter within a sheath, in accordance with various embodiments of the present disclosure. A catheter 60A includes a sheath 62 (i.e., the sheath may be integrated with the catheter 60) surrounding a lumen 64. The catheter 60A may furcate into a plurality of catheterlets 66, where the plurality of catheterlets 66 are located in the lumen 64 of the sheath 62. In some embodiments, the lumen 64 may comprise the entire interior of the sheath 62. Other embodiments may comprise a lumen smaller than the entire interior of the sheath 62 and/or multiple lumens within the sheath 62. In some embodiments, the sheath 62 can be separate from the catheter 60A (e.g., catheter 60A can be used with various sheaths/introducers).

In the embodiment shown in FIG. 2A, sheath 62 can be, for example, a 10 french (fr) inside diameter (ID) and each of a plurality of catheterlets 66 can be 2 fr in diameter. Such an embodiment would allow 17 catheterlets 66 inside the lumen 64 of sheath 62. Other configurations are possible, including different sizes for the sheath 62 (e.g., inner diameter is larger or smaller than 10 french) and different sizes for the plurality of catheterlets 66 (e.g., larger or smaller than 2 french). An outside diameter 68 of the sheath 62 can be any suitable size, including outer diameters ranging from 8.5-13 fr (approximately 2.834-4.333 mm).

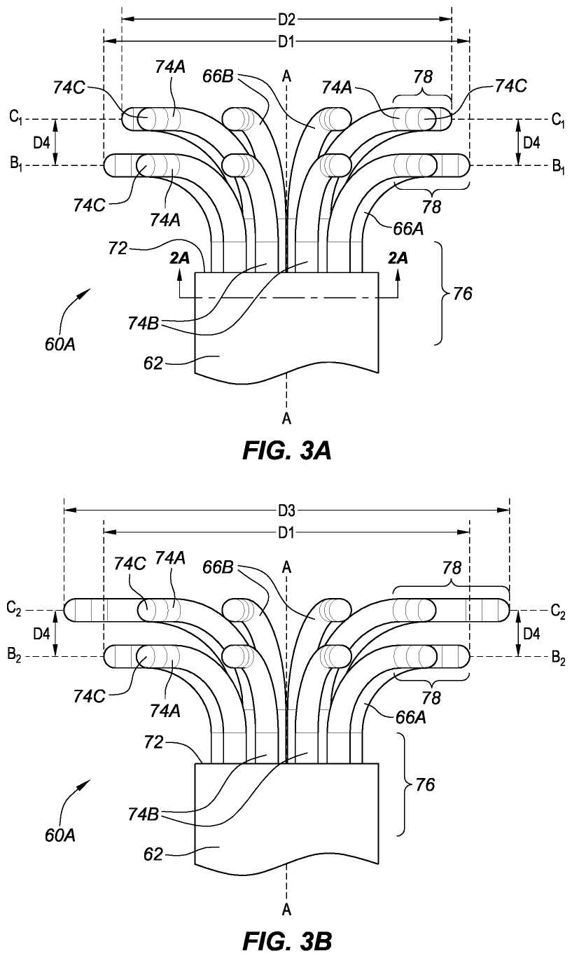

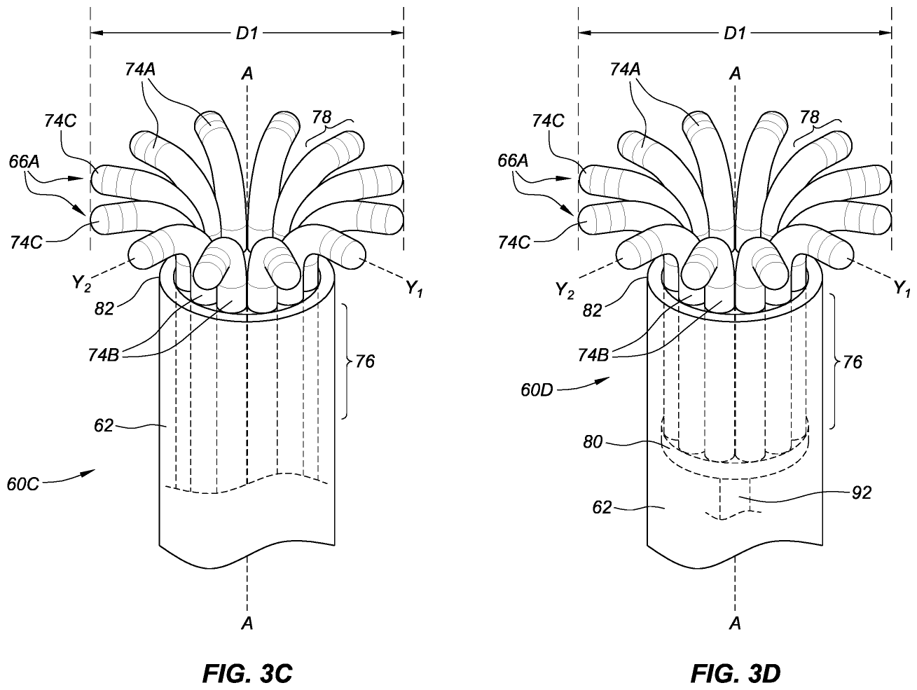

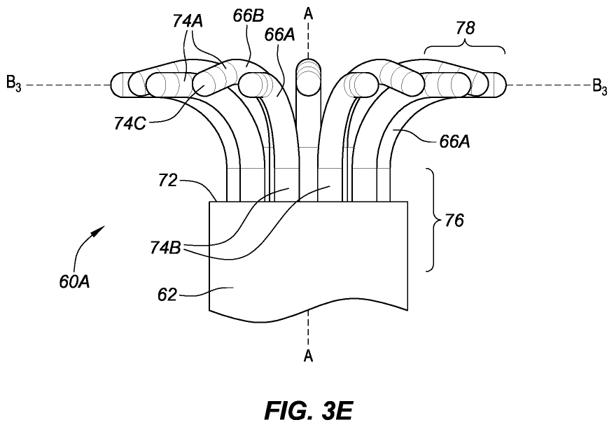

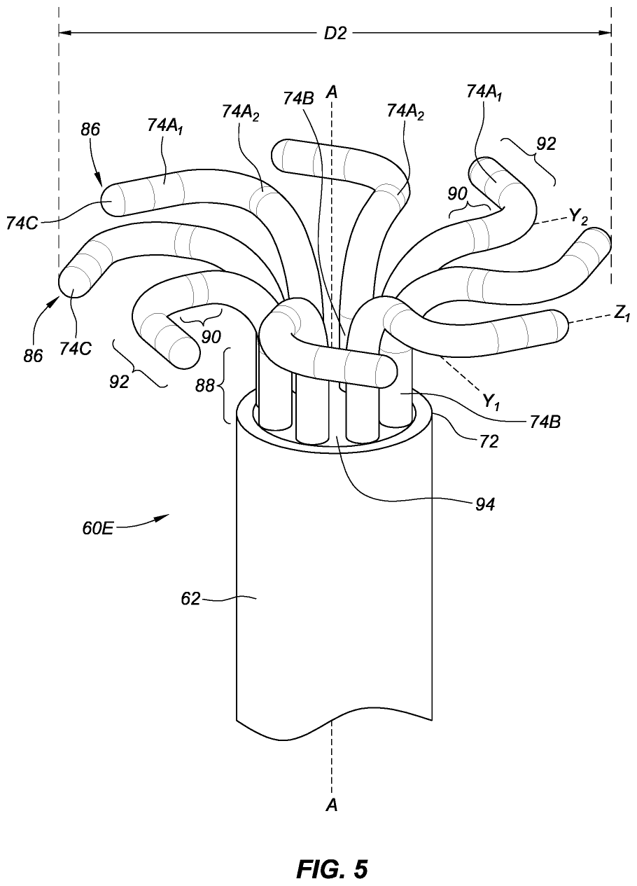

The plurality of catheterlets 66 can also be arranged in different patterns in lumen 64 of sheath 62. In various embodiments, the plurality of catheterlets 66 are divided into two groups, a first plurality of catheterlets 66A and a second plurality of catheterlets 66B. FIG. 2A shows the first plurality of catheterlets 66A placed in a ring adjacent to sheath 62 (e.g., proximate an inner wall of the sheath) and the second plurality of catheterlets 66B encircling a longitudinal axis of the sheath 62. In another embodiment (not shown), the plurality of catheterlets 66 may include a first plurality of catheterlets 66A placed in a ring adjacent to the sheath (as shown in FIG. 2A), but within the second plurality of catheterlets 66B. Such a configuration allowing additional room in the lumen 64 (see, e.g., FIGS. 3C-D) for other items such as a single central catheter (e.g., with a diameter larger than the each of the plurality of catheterlets 66 as in FIG. 5, one or more lead wires, steering wires, sensors, irrigant lumens, etc. In some embodiments, the first plurality of catheterlets 66A and the second plurality of catheterlets 66B can be moved independently (e.g., the first and/or the second plurality of catheterlets can be advanced/retracted independently with respect to the other). Still other embodiments can include one or more catheterlets and/or groups of catheterlets being channeled through a plurality of lumens in the sheath 62 (e.g., to prevent tangling between the various catheterlets)

One or more of the plurality of catheterlets 66 may include electrodes in proximity to a distal tip (see FIGS. 3A-D, 5, and 6 and related discussion). In some embodiments, each catheterlet may have the same or a unique number of electrode with respect to the other catheterlets.

Some or all of the catheterlets 66 can include an irrigation port (not shown) at various locations such as at the electrode, proximate the electrode, through the electrode, and/or proximate the point of furcation of each of the plurality of catheterlets 66. A fluid can be circulated through an irrigant lumen and out through the irrigation ports.

FIG. 2B is a cross-sectional view of multiple catheterlets 66A of a catheter 60B within a sheath surrounding a central catheterlet 70, in accordance with various embodiments of the present disclosure. The catheter 60B includes a sheath 62 surrounding a lumen 64. The catheter 60B can furcate into a plurality of catheterlets 66 and a central catheterlet 70.

FIGS. 3A-B are side views of a distal end of a catheter of FIG. 2A with a plurality of catheterlets deployed. FIG. 3A shows a plurality of inner catheterlets radially deployed less than a plurality of outer catheterlets. FIG. 3B shows a plurality of inner catheterlets radially deployed further than a plurality of outer catheterlets are deployed. The location of a cross section of the catheter 60A as shown in FIG. 2A is indicated in FIG. 3A.

Catheter 60A includes a sheath 62 with a distal end 72 and a plurality of catheterlets 66A and a plurality of catheterlets 66B extending out from distal end 72. A longitudinal axis of catheter 60A is defined by line A-A, and at least a portion of the plurality of catheterlets 66 substantially extending along the longitudinal axis A-A.

The plurality of catheterlets 66A/66B can be extended and retracted with respect to distal end 72 of sheath 62. The plurality of catheterlets 66A/66B can be connected (directly or indirectly) to a control mechanism (e.g., a manual control mechanism such as, for example, the handle 24 (FIG. 1), a robotic control mechanism, or some other control interface) that facilitates extension/retraction of the catheterlets. The plurality of catheterlets 66A/66B can comprise a flexible material and structure, which facilitates conformance to various tissue geometries (e.g., complex endocardial topologies such as the antrum of pulmonary veins) contacted therewith.

Each of the plurality of catheterlets 66A/66B may include one or more electrodes 74A, 74B, 74C. The electrodes can be used for mapping anatomical features and/or delivering therapy to contacted tissue. Mapping and therapy can occur independently or simultaneously (e.g., some electrodes can be used to map while other electrodes delivery therapy).

When the plurality of catheterlets 66A/66B are deployed from the catheter 60A, the electrodes 74A may contact tissue. The one or more electrodes 74B, also extended out of the catheter may conduct non-contact electrophysiology mapping.

Catheter 60A of FIG. 3A-B may simultaneously conduct on the catheterlets 66A/B linear ablation and multi-electrode mapping. Moreover, the plurality of electrodes on the catheterlets facilitate faster mapping of anatomical structures and electrophysiology characteristics.

Catheterlets 66A/66B can take on multiple shapes based on their position relative to sheath 62 of catheter 60A. Movement of the sheath 62, with respect to the plurality of catheterlets 66A/66B, may facilitate different configurations of the plurality of catheterlets 66A/66B.

In one embodiment, a distal portion of each of the plurality of catheterlets 66A/66B can form an “L” shape when extended beyond sheath 62. A first distal portion 76 of each of the plurality of catheterlets 66A/66B extend substantially parallel with the longitudinal axis A-A, and a second distal portion 78 extends perpendicular to the longitudinal axis A-A. That is, the second distal portion 78 of catheterlet 66A extends along line B₁-B₁, and the second distal portion of catheterlets 66A/66B extend along line C₁-C₁.

Other configurations of the plurality of catheterlets 66A/66B are possible when different ones of the, or lesser amounts of the distal portion of the, plurality of catheterlets are extended from the sheath 62. When the plurality of catheterlets 66A/66B are extended from the sheath 62 enough to allow contact between each of the plurality of catheterlets 66A and/or the plurality of catheterlets 66B and tissue, the radius of coverage is essentially the radius of the outermost catheterlets (e.g., the outer row of catheterlets 66A/66B shown in FIGS. 2A-B). As the plurality of catheterlets 66A/66B are further extended out of the sheath 62, the distal portion of each of the plurality of catheterlets 66A/66B begin to curve. This curvature may cause the angle between a first longitudinal axis A-A of a first distal portion 76 and second longitudinal axis of a second distal portion 78 (defined by line B₁-B₁, for the plurality of catheterlets 66A and line C₁-C₁ for the plurality of catheterlets 66B) to change (e.g., increase from 0°). For example, the angle between the first longitudinal axis A_{X}-A_{X} and the second longitudinal axis B₁-B₁, or C₁-C₁ may be 0-90° as the catheterlets extend. A maximum angle, approximately 90°, being met when the radius of coverage is at a maximum, as shown in FIGS. 3C-D.

The plurality of catheterlets 66A/66B can have a pre-set curvature. The pre-set curvature can be the same for each of the plurality of catheterlets 66A/66B or can vary for one or more of the plurality of catheterlets 66A/66B. The pre-set curvature can be formed by an element in each catheterlet that induces a curve in the catheterlet after extension from the sheath 62, such as a piece of wire, a strip of material with shape memory (e.g., Nitinol). The pre-set curvature can allow the plurality of catheterlets 66A/66B to form a specific angle when extended from the sheath 62. As described above, one embodiment can have a pre-set curvature that generates an angle of 90° between a longitudinal axis A-A of the first distal portion 76 and a second longitudinal axis B₁-B₁, or C₁-C₁ of a corresponding second distal portion 78 of a catheterlet. Other angles are possible as described above.

In some embodiments, each of the plurality of catheterlets 66A/66B can be individually controlled. For example, each of the plurality of catheterlets 66A/66B can have a separate control mechanism (e.g., one or more pull wires, sliding connector, etc.) (not shown). The separate control mechanisms can control, for example, the longitudinal movement and/or the curvature of each of the plurality of catheterlets 66A/66B individually (e.g., each of catheterlets can be advanced/retracted a different distance from the sheath).

In other embodiments, the plurality of catheterlets 66A/66B can be controlled in groups by a group control device (not shown, see FIG. 3D and related discussion). For example, a ring or other similar device could be connected, directly or indirectly, to a proximal end portion of the plurality of catheterlets 66A/66B. The group control device could advance distally and/or retract proximally a group of the plurality of catheterlets 66A/66B by manipulating the group control device (e.g., tilting, pivoting, etc.). This manipulation of the control device could allow a portion of the plurality of catheterlets 66A/66B to be moved distally and proximally. More than one group control device could be used with each device controlling a portion of the plurality of catheterlets (e.g., two group control devices, with each controlling 50% of the catheterlets, four group control devices, with each controlling 25% of the catheterlets, etc.) Control of the group control device could be done by, for example, a user (e.g., a physician or other clinician) or by a robotic mechanism.

When deployed, the plurality of catheterlets 66A can have a diameter of D₁ that can vary (e.g., depending on how far the plurality of catheterlets are extended from the sheath 62 and/or the curve (pre-set or variable through a control mechanism as described herein)). Similarly, when deployed, the plurality of catheterlets 66B can have a diameter of D₂ that can vary. In the configuration shown in FIG. 3A, the diameter D₁ is larger than the diameter D₂. Embodiments where the catheterlets are individually controllable could allow for additional variations of the diameter of the plurality of catheterlets 66A and 66B. In the configuration shown in FIG. 3B, the diameter D₁ is smaller than the diameter D₂. Some embodiments (not shown) can have diameters of the plurality of catheterlets 66A/66B being equal (e.g., D₁ equal to the D₂ and D₁ equal to D₃) and the distance D₄ is greater than zero. Other embodiments can have the distance D₄ effectively zero (see, e.g., FIG. 3E and related discussion).

Distal portions 78 of the plurality of catheterlets 66A/66B can be separated by a distance D₄ (as measured along the longitudinal axis A-A. The distance D₄ can be fixed or it can vary, depending on how the plurality of catheterlets 66A/66B are controlled. Where individual catheterlets are controllable, the distance D₄ can vary within a group of catheterlets (e.g., D₄ can be different for one or more of the plurality of catheterlets 66A/66B).

The plurality of catheterlets 66A/66B accommodate complex endocardial topologies such as an antrum of the pulmonary veins. Catheters with other designs cannot allow for similar variations in topologizes while maintaining consistent contact. The adjustability of the plurality of catheterlets 66A/66B can allow for “one-shot” treatment of tissue. For example, create an ablation line that is continuous around an anatomical location in contact with the plurality of catheterlets 66A, such as an antrum of a pulmonary vein. The one-shot treatment can occur when the plurality of catheterlets 66A are partially or fully deployed (i.e., extended) from the catheter. The adjustability of the plurality of catheterlets 66A/66B can also allow for one-shot irreversible electroporation (IRE). Aspects of the present disclosure benefit from improved contact with tissue and easier placement compared to other catheters that use, for example, a spiral, a basket or a balloon. The ablation energy and delivery technology used on the present disclosure may include, by way of example and without limitation, one or more of the following: cryogenic, RF, laser, microwave, ultrasound (including high intensity focused ultrasound) and microwave.

Deploying multiple catheterlets that all make contact with tissue (e.g., the antrum of pulmonary veins), stabilizes the entire assembly, and can reduce the likelihood of the catheter moving during therapy. For example, a first portion of the plurality of catheterlets can be positioned in contact with tissue that is not targeted for treatment, while a second portion of the plurality of catheterlets can be positioned in contact with targeted tissue. Improved stability due to multiple contact points between the catheter and tissue is possible, for example, at the carina between the left superior pulmonary vein and the left atrial appendage.

The configurations shown in FIGS. 3A-B can allow for contact with tissue in various configurations. For example, the plurality of catheterlets 66A/66B can allow for a “double lasso” technique where the plurality of catheterlets 66A contact tissue in one area and the plurality of catheterlets 66B contacts tissue in another area (e.g., proximate the pulmonary veins to detect entrance and exit block in conjunction with conducting pulmonary vein isolation ablation).

One or more of the plurality of catheterlets 66A/66B can have an aspect ratio (e.g., elliptical or rectangular cross section) that can provide greater lateral stability. The increase in stability can aid in creating more uniform separation distance between each of the electrodes 74 on the plurality of catheterlets 66A/66B which is beneficial for pulmonary vein isolation where avoidance of lesion gaps is desirable.

FIGS. 3C-D are isometric distal end views of a catheter with multiple catheterlets deployed, in accordance with embodiments of the present disclosure. A catheter 60C can include a sheath 62 with a distal end 76 from which a plurality of catheterlets 66A extend. The catheter 60C has a longitudinal axis defined by the line A-A, and the plurality of catheterlets 66A can each have a portion extending parallel to the longitudinal axis A-A, and another portion extending non-parallel (when deployed).

The plurality of catheterlets 66A can be extended and retracted with respect to the distal end 76 of the sheath 62. The plurality of catheterlets 66A can be connected (directly or indirectly) to a control mechanism (e.g., a manual control mechanism such as, for example, handle 24 of FIG. 1, a robotic control mechanism, or some other control interface). The plurality of catheterlets 66A can comprise a flexible material which facilitates conformance to various tissue topologizes (e.g., complex endocardial topologies such as an antrum of a pulmonary vein). The movement of the plurality of catheterlets 66A can be analogous to the movement of the tentacles of a sea anemone.

Each of the plurality of catheterlets 66A can include one or more electrodes 74A, 74B, 74C. The one or more electrodes 74A, 74B, 74C can be used for mapping anatomical features and/or delivering therapy to tissue. Mapping and therapy can occur individually or at the same time. For example, some electrodes 74B can be used to map while other electrodes 74A, 74C delivery therapy.

When the plurality of catheterlets 66A are extended from the distal end 76 of the catheter 60C, electrodes 74A can be positioned for contact with tissue. The one or more electrodes 74B can be used for functions that do not require contact with tissue (e.g., catheter localization and non-contact electrophysiology mapping). Additional electrodes (e.g., 74A₁, 74A₂) can be included in various other embodiments to provide additional tissue contact locations for therapy (e.g., see FIG. 5 and related discussion for more information). Similarly, additional electrodes can be included in some embodiments for additional non-contact functions.

The electrodes 74A, 74B on each of the plurality of catheterlets 66A allow the catheter 60C to be used as a multi-purpose device that can simultaneously act as a linear ablation catheter and electrophysiology mapping catheter. Additional electrodes (see FIG. 5) can be added to the catheterlets to sense electrode data at multiple points of tissue contact which allows for faster/higher mapping of anatomical structures.

The plurality of catheterlets 66A can take on multiple shapes based on their position relative to sheath 62. Movement of the sheath 62 with respect to the plurality of catheterlets 66A can achieve various configurations of the catheterlets 66A.

In one embodiment, a distal portion of each of the plurality of catheterlets 66A can form an “L” shape when fully extended beyond sheath 62. In this embodiment, the distal portions of each of the plurality of catheterlets 66A can have a first distal portion 76 that is generally parallel with a longitudinal axis of catheter 60C (defined by the line A-A) and a second distal portion 78 that extends perpendicular to the sheath 62 when the catheterlet is fully extended (defined by the line Y_{X}-Y_{X}, where x represents a different number for each of the longitudinal axes for each of the catheterlets). This configuration creates an angle of approximately 90 between the first longitudinal axis for the first distal portion 76 and the second longitudinal axis for the second distal portion 78.

Other configurations of the plurality of catheterlets 66A are possible when different, lesser, amounts of the distal portion of the plurality of catheterlets are extended from the sheath 62. When the plurality of catheterlets 66A are extended from the sheath 62 just enough to allow contact between each of the plurality of catheterlets 66A and tissue, the radius of coverage is essentially the radius of the sheath 62. As the plurality of catheterlets 66A are further extended out of the sheath 62, the distal portion of each of the plurality of catheterlets 66A extending from the sheath 62 can begin to curve. This curvature causes the angle between the first longitudinal axis A-A of the first distal portion 76 and second longitudinal axis of the second distal portion 78 (defined by the line Y_{X}-Y_{X}) to change (e.g., increase from 0°). For example, the angle between the first longitudinal axis A-A of the first distal portion 76 and the second longitudinal axis Y_{X}-Y_{X} of the corresponding second distal portion 78 can be between 0-90°, for example as the catheterlets extend until the radius of coverage is at a maximum.

The plurality of catheterlets 66A can have a pre-set curvature. The pre-set curvature can be the same for each of the plurality of catheterlets 66A or can vary for one or more of the plurality of catheterlets 66A. The pre-set curvature can be formed by an element in each catheterlet that induces a curve in the catheterlet after extension from the sheath 62. The pre-set curvature can allow the plurality of catheterlets 66A to form a specific angle.

In some embodiments, each of the plurality of catheterlets 66A can be individually controlled. For example, each of the plurality of catheterlets 66A can have a separate control mechanism (e.g., one or more pull wires, sliding connector, etc. The separate control mechanisms can control, for example, the longitudinal movement and/or the curvature of each of the plurality of catheterlets 66A individually.

In some embodiments, a plurality of catheterlets 66A can be controlled in groups by a group control device. For example, a ring or other similar device could be connected, directly or indirectly, to a proximal end portion of a group of the plurality of catheterlets 66A. The group control device could advance (e.g., distally) and/or retract (proximally) a group of the plurality of catheterlets 66A by manipulating the group control device (e.g., tilting, pivoting, etc.). This manipulation of the control device could allow a portion of the plurality of catheterlets 66A to be moved distally and proximally. More than one group control device could be used with each device controlling a portion of the plurality of catheterlets (e.g., two group control devices, with each controlling 50% of the catheterlets, four group control devices, with each controlling 25% of the catheterlets, etc.) Control of the group control device could be done by, for example, a user (e.g., a physician or other clinician) or by a robotic mechanism.

The plurality of catheterlets 66A can accommodate complex endocardial topologies such as an antrum of a pulmonary vein. Catheters with other designs cannot allow for similar variations in shape. The adjustability of the plurality of catheterlets 66A can allow for “one-shot” treatment of tissue (e.g., a single instance of therapy) to, for example, create an ablation line that is continuous around an anatomical location that is in contact with the plurality of catheterlets 66A, such as the antrum of pulmonary veins. The one-shot treatment can occur when the plurality of catheterlets 66A are partially or fully deployed (i.e., extended) from the catheter. The adjustability of the plurality of catheterlets 66A can also allow for one-shot irreversible electroporation (IRE) and can provide better contact with tissue and easier placement compared to other catheters that use, for example, a spiral, a basket or a balloon to make contact with tissue and/or deliver therapy to target tissue.

Deploying multiple catheterlets that all make contact with tissue (e.g., the antrum of pulmonary veins) stabilizes the entire assembly and can reduce the likelihood of unintentional catheter movement during diagnosis and therapy. For example, a first portion of the plurality of catheterlets can be positioned to be in contact with tissue that is not targeted for treatment while a second portion of the plurality of catheterlets can be positioned to be in contact with tissue that is targeted for treatment.

One or more of the plurality of catheterlets 66A can have an aspect ratio (e.g., elliptical or rectangular cross section) that can provide greater lateral stability. The increase in stability can aid in creating more uniform separation between each of the electrodes 74 on the catheterlets 66A, which is beneficial for pulmonary vein isolation where avoidance of lesion gaps is a priority.

FIGS. 3C and 3D show different configurations for a plurality of catheterlets within a catheter. In FIG. 3C, a plurality of catheterlets 66A can extend from a distal end 82 to a proximal end of a catheter 60C. This configuration can allow for, among other things, individual control of movement for each of the catheterlets 66A. In FIG. 3D, a plurality of catheterlets 66A can extend from a distal end 82 of a catheter 60D to an intermediate location within sheath 62. The intermediate location can be at any location between the distal end 82 and the proximal end of the catheter 60D. In the embodiment shown in FIG. 3D, the intermediate location is proximate the distal end 82 which uses less material than an intermediate location positioned more proximally.

The intermediate location can have a connector 80 that couples the plurality of catheterlets 66A with an elongate device 92 (e.g., a wire, or a connecting linkage). The elongate device 92 controls the longitudinal movement of catheterlets 66A with respect to the catheter 60D. The elongate device 92 can be connected (directly or indirectly) to a control mechanism in the handle 24 (FIG. 1), or a robotic control mechanism, or some other control interface. The connector 80 can be any suitable shape including a ring, disk, etc. The connector 80 can be coupled with the plurality of catheterlets 66A using any suitable method (e.g., adhesive, crimping, swaging, etc.).

FIG. 3E is a distal, side view of a catheter 60A with multiple interleaved catheterlets deployed, with inner and outer catheterlets are deployed, and the distal ends of the plurality of catheterlets are generally planar, in accordance with various embodiments of the present disclosure.

A plurality of catheterlets 66A/66B can be arranged, when deployed, so that the inner and outer catheterlets are interleaved (i.e., the inner and outer catheterlets are radially alternating about the circumference of the catheter when deployed).

Each of the plurality of catheterlets 66A/66B can be similar to those described and shown in reference to FIGS. 3A-D with one or more electrodes 74A, 74B, 74C that can be used for mapping anatomical features and/or delivering therapy to tissue.

When the plurality of catheterlets 66A/66B are deployed as shown in FIG. 3E, a first distal portion 76 of each catheterlet can be generally parallel with a longitudinal axis of a catheter 60A (defined by line A-A) and a second distal portion 78 generally perpendicular to A-A when the catheterlet is fully extended. Second distal portion 78 extending along a line B₃—B₃, for the plurality of catheterlets 66A/66B. The second distal portions 78 of each of the plurality of catheterlets 66A/66B are essentially planar.

In the catheter disclosed in FIG. 3E, the plurality of catheterlets 66A/66B equally extend radially outward from sheath 62. In some embodiments, one or more of the plurality of catheterlets could extend radially outward further than other catheterlets, but still be generally planar with the other plurality of catheterlets.

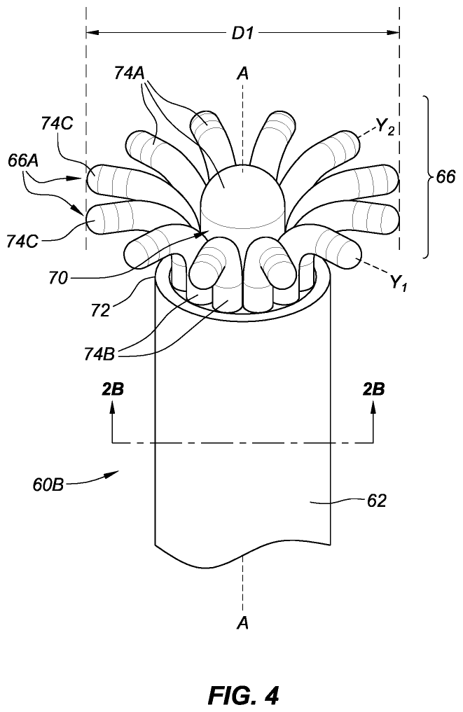

FIG. 4 is an isometric distal end view of a catheter 60B with multiple catheterlets 66A surrounding a central catheterlet 70 as shown in FIG. 2B, in accordance with various embodiments of the present disclosure. The catheter 60B includes a sheath 62 with a distal end 72 and a proximal end (not shown; see FIG. 1), and a plurality of catheterlets 66A surrounding a central catheterlet 70 at the distal end 72 of the catheter 60B. The catheter 60B having a longitudinal axis defined by line A-A and a proximal portion of the plurality of catheterlets 66A extending along the longitudinal axis A-A. The location of a cross section 2B-2B of the catheter 60B, as shown in FIG. 2B, is indicated in FIG. 4.

The plurality of catheterlets 66A and the central catheterlet 70 can be extended (i.e., deployed) and retracted with respect to distal end 72 of catheter 60B. The extent of deployment of the plurality of catheterlets 66A results in various shapes as described herein with reference to FIGS. 3A-D.

The catheter 60B and/or the plurality of catheterlets 66A can be connected (directly or indirectly) to a control mechanism (e.g., a manual control mechanism on a handle, a robotic control mechanism, or some other control interface). The plurality of catheterlets 66A and the central catheterlet 70 can comprise a flexible material structure that facilitates conformance to various tissue configurations (e.g., complex endocardial topologies such as the antrum of pulmonary veins).

Each of the plurality of catheterlets 66A and the central catheterlet 70 can include one or more electrodes (74A, 74B, 74C). The electrodes can be used for mapping anatomical features, diagnosis, and/or delivering therapy to tissue. Diagnosis and therapy can occur in series of in parallel (e.g., some electrodes 74A can be used to sense electrophysiological characteristics of the tissue map while other electrodes 74A delivery therapy).

When the plurality of catheterlets 66A are extended from distal end 72 of catheter 60B, electrodes 74A may be placed in contact with tissue. The electrodes 74B can also be positioned outside the distal end 72 of the catheter 60B and used for functions that do not require contact with tissue (e.g., mapping catheter location and non-contact electrophysiology sensing). Additional electrodes 74A may provide additional tissue contact locations for therapy.

The electrodes 74A, 74B on each of the catheterlets 66A and central catheterlet 70 facilitate use of the catheter 60B as a multi-purpose device that can simultaneously provide real-time electrophsyiology sensing and linear ablation therapy.

The embodiment shown in FIG. 4 facilitates improved stability and continuous contact with target tissue.

FIG. 5 is an isometric, distal end view of a catheter 60E with a plurality of compound catheterlets 86, in accordance with various embodiments of the present disclosure. The catheter 60E includes a sheath 62 and a plurality of compound catheterlets 86 at a distal end 72 of the sheath 62. Similar to FIGS. 3A-D, and 4, the plurality of compound catheterlets 86 can be extended and retracted with respect to the distal end 72 of the sheath 62. The catheter 60E and/or the plurality of compound catheterlets 86 can be connected (directly or indirectly) to a control mechanism (e.g., a manual control mechanism such as, for example, a catheter handle, a robotic control mechanism, or some other control interface).

Each of the plurality of compound catheterlets 86 are formed into two or more curved portions. The curved portions facilitating desired configuration of the plurality of catheterlets 86. For example, it has been discovered that the embodiment of FIG. 5 facilitates optimal contact with tissue. Another benefit of the present embodiment is that the multiple portions of each catheterlet can contact tissue, reducing the number of catheterlets needed for a specific application.

In one embodiment, a distal portion of each of the plurality of compound catheterlets 86 can form an “S” shape when the distal portions are extended beyond the sheath 62. In the embodiment of FIG. 5, the distal portions of each of the plurality of catheterlets 86 have a first distal portion 88 parallel with a longitudinal axis A-A of sheath 62, a second distal portion 90 with a second longitudinal axis (defined by the line Y_{X}-Y_{X}, where X represents a unique axes for each of the catheterlets) that is generally perpendicular to axis A-A when the catheterlet is fully extended, and a third distal portion 92 with a third longitudinal axis (defined by the line Z_{X}-Z_{X}, where X represents a unique axes for each of the catheterlets that, like the second distal portion 90 is also perpendicular to the first longitudinal axis, and at an angle with respect to the second longitudinal axis (e.g., 900 as shown in FIG. 5). This configuration can create an angle of approximately 90° between the first longitudinal axis of the first distal portion 88 and the second longitudinal axis Y_{X}-Y_{X} of the second distal portion 90, and an angle of 90° between the second longitudinal axis Y_{X}-Y_{X} of the second distal portion 90 and the third longitudinal axis Z_{X}-Z_{X} of the third distal portion 92.

Other configurations of the plurality of compound catheterlets 86 are possible when different, lesser, catheterlets 86 are arranged in the sheath 62. When the plurality of catheterlets 86 are extended from the sheath 62 just enough to allow contact between each of the plurality of catheterlets 66A and tissue, the radius of coverage is essentially the radius of the sheath 62. As the plurality of compound catheterlets 86 are further extended out of the sheath 62, the distal portion of each of the plurality of compound catheterlets 86 extending from the sheath 62 can begin to curve in multiple directions. This curvature can cause the angle between the first distal portion 88 and the second distal portion 90, and the third distal portion 92 to deviate from their parallel configuration within the sheath. For example, the angle between the first distal portion 88 and the second distal portion 90 and the angle between the second distal portion 90 and the third distal portion 92 can be between 0-90° as the compound catheterlets extend until the radius of coverage is at a maximum and the angle between the first distal portion 88, the second distal portion 90, and the third distal portion 92 of the plurality of compound catheterlets is approximately 90°, as shown in FIG. 5.

The plurality of compound catheterlets 86 can have a pre-set curvature. The pre-set curvature can be the same for each of the plurality of compound catheterlets 86 or can vary for one or more of the plurality of compound catheterlets 86 or catheterlet portions of each catheterlet. The pre-set curvature can allow the plurality of compound catheterlets 86 to form a specific angle. As described above, one embodiment can have a pre-set curvature that generates an angle of 90° between the first distal portion and the second distal portion and an angle of 90° between the second distal portion and the third distal portion of a compound catheterlet. Various other angles are readily envisioned.

Each of the plurality of compound catheterlets 86 in FIG. 5 can include one or more electrodes 74A₁, 74A₂, 74B, and 74C. The one or more electrodes can be used for electrophysiology mapping of anatomical features and/or delivering therapy to tissue. Mapping and therapy can occur individually or simultaneously.

The one or more electrodes 74A₁, 74A₂, 74B, and 74C on each of the catheterlets 86 facilitate increased sensing resolution of electrogram data which allows for faster/more accurate mapping of anatomical structures. The one or more electrodes on each of the plurality of compound catheterlets can also be used to sense multiple electrograms in an unorganized array of Orientation Independent Sensing (OIS) maps.

Any of the catheter embodiments discussed herein may include a spacer plate. For example, FIG. 5 includes a spacer plate 94 that can be used to maintain a specific spacing between each of the plurality of compound catheterlets 86. The spacer plate 94 can be coupled with the sheath 62 at or proximate a distal end 72. The spacer plate 94 can also assist with preventing/limiting blood or other items from ingressing into entering the distal end 72 of the sheath 62, or otherwise inhibiting movement of the catheterlets. This spacer plate 94 may also help maintain smooth and unobstructed movement of the catheterlets.

The spacer plate 94 can also include one or more irrigant apertures connected to a fluid source for delivering fluid to a distal end 72 of sheath 62.

Any of the catheterlets described herein can incorporate sensors/elements to detect and measure contact with tissue. For example, an electrical coupling index (ECI) value can be used to determine tissue contact when providing therapy (e.g., ablation). Another element can be mechanical deformation sensors (e.g., TactiSys™/TactiCath™, ultrasound, or other techniques to ensure effective tissue mapping and therapy (e.g., lesion delivery, etc.). Still another sensor that can be incorporated (not shown) is a shape sensor (e.g., a fiber optic shape sensor) that can provide information regarding a curvature of a catheter when deflected (which can translate to a force imposed on the catheter).

For example, a force sensing (i.e., contact force) system and force sensor (not shown) may include technology similar to or the same as that used in the TactiCath™ Quartz™ Ablation Catheter system, commercially available from St. Jude Medical, Inc. of St. Paul Minn. Additionally, or alternatively, the force sensing system and force sensor may include force sensing sensors, systems, and techniques illustrated and/or described in one or more of U.S. patent application publication nos. 2007/0060847; 2008/0009750; and 2011/0270046, each of which is hereby incorporated by reference in its entirety as though fully set forth herein.

An embodiment similar to the system of FIG. 1 may be used to determine the ECI value, for example, and as described in detail in U.S. Pat. No. 8,403,925, which is incorporated by reference herein in its entirety as though fully set forth herein. Additional information about ECI and lesion monitoring is described in U.S. patent application publication nos. 2011/0144524, 2011/0264000, and 2013/0226169, each of which is hereby incorporated by reference as though fully set forth herein.

Any of the catheterlets described herein may incorporate a magnetic sensor. The magnetic sensor may facilitate, for example, precise placement/annotation of ablation lesions and prediction of gaps between ablation lesions (e.g., using the magnetic field generator 52 shown in FIG. 1 and described above).

Although at least one embodiment of an apparatus with multiple catheterlets for sensing, mapping, and providing therapy has been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and can include intermediate members between a connection of elements and relative movement between elements and can also include elements that are part of a mixture or similar configuration. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure can be made without departing from the spirit of the disclosure as defined in the appended claims.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A catheter comprising:
a plurality of flexible catheterlets, each catheterlet including a proximal end, a distal end, and an electrode proximate the distal end; and wherein a tip of the catheterlet is free of electrodes.

2. The catheter of claim 1, further including a sheath, wherein the plurality of catheterlets are configured and arranged to move relative to the sheath.

3. The catheter of claim 2, wherein a portion of the plurality of catheterlets are arranged in a ring positioned proximate an inner wall of the sheath.

4. The catheter of claim 3, further including a central catheterlet, wherein the plurality of catheterlets circumferentially extend about the central catheterlet.

5. The catheter of claim 4, wherein a diameter of the central catheterlet is larger than a diameter of the plurality of catheterlets.

6. The catheter of claim 3, wherein the plurality of catheterlets are configured and arranged to contact and thereby stabilize contact between the central catheterlet and the tissue.

7. (canceled)

8. The catheter of claim 2, wherein each of the plurality of catheterlets are configured and arranged to extend out of the sheath, and deploy a first portion of the catheterlets along a first longitudinal axis and a second portion along a second longitudinal axis, where the first longitudinal axis is substantially parallel with a catheter longitudinal axis and where an angle between the first longitudinal axis and the second longitudinal axis is between 0° and 90°.

9. The catheter of claim 1, wherein a portion of the plurality of catheterlets are coupled with a group control device, wherein the group control device is configured and arranged to control one or more of a longitudinal movement and a curvature of a distal end of the plurality of catheterlets in the portion.

10. The catheter of claim 1, wherein a portion of the plurality of catheterlets are coupled with a connector at an intermediate location between a distal end and a proximal end of the catheter, and where the connector is coupled with a control element configured and arranged to extend proximally to a proximal location of the elongate medical device.

11. (canceled)

12. The catheter of claim 2, wherein the plurality of catheterlets are coupled with one or more control elements configured and arranged to control a shape of the corresponding catheterlet distal end and/or to control longitudinal movement of the corresponding catheterlet with respect to the sheath.

13. (canceled)

14. The catheter of claim 1, wherein a first portion of the plurality of catheterlets are configured and arranged to form a first diameter when deployed, and a second portion of the plurality of catheterlets are configured and arranged to form a second diameter when deployed, wherein the first diameter is different than the second diameter.

15. (canceled)

16. (canceled)

17. The catheter of claim 1, wherein a portion of the plurality of catheterlets are configured to determine one or more of a contact force and an electrical coupling index.

18. (canceled)

19. (canceled)

20. A catheter comprising:
a plurality of catheterlets including
a first portion with a first longitudinal axis,
a second portion with a second longitudinal axis, and
a third portion with a third longitudinal axis; and
wherein the catheterlets are configured and arranged to have a deployed position and an undeployed position,
in the undeployed position, the first longitudinal axis, the second longitudinal axis, and the third longitudinal axis are each substantially parallel with a catheter longitudinal axis, and
in the deployed position, the first longitudinal axis is substantially parallel with the catheter longitudinal axis, the first longitudinal axis has a first angle relative to the second longitudinal axis, and the second longitudinal axis has a second angle relative to the third longitudinal axis when the catheterlets are partially or fully extended from a sheath; and
wherein a distal end of each of the plurality of catheterlets is unsecured.

21. The catheter of claim 20, wherein each the plurality of catheterlets include
a proximal end,
the distal end,
a first electrode proximate the distal end,
a second electrode is proximal of the first electrode, and
an angled portion between the first and second electrodes configured and arranged to bend, and thereby form an angle, in response to deployment of the catheterlet from the sheath.

22. The catheter of claim 20, wherein the first angle is between 0° and 900 and the second angle is between 0° and 900 when the catheterlets are partially or fully extended from the sheath

23. A catheter comprising:
a central catheterlet including a first electrode;
a plurality of peripheral catheterlets, each of the peripheral catheterlets include at least one electrode; and
wherein the plurality of peripheral catheterlets are circumferentially positioned about the central catheterlet.

24. The catheter of claim 23, wherein a diameter of the central catheterlet is larger than a diameter of each of the peripheral catheterlets.

25. The catheter of claim 23, further including a sheath, wherein the central catheterlet and the plurality of peripheral catheterlets are configured and arranged to be stored within the sheath, and to move relative to the sheath along a common longitudinal axis.

26. The catheter of claim 25, wherein the central catheterlet is configured and arranged to be controlled by a first control element and the plurality of peripheral catheterlets are configured and arranged to be controlled by a second control element, where the first control element controls a shape of the central catheterlet distal end and the second control element each control a shape of the corresponding peripheral catheterlet distal end.

27. The catheter of claim 25, wherein the central catheterlet and the plurality of peripheral catheterlets are further configured and arranged to extend into a deployed position in response to exiting the sheath, in the deployed position the plurality of catheterlets are positioned for contact with tissue and to thereby increase stability of the central catheterlet relative to the tissue.
